(19) United States
(12) Patent Application Publication
(10) Pub. No.: US 2023/0270716 A1
(43) Pub. Date: Aug. 31, 2023

(54) GLYCOPYRROLATE ORAL FILM AND IT'S PROCESS

(71) Applicant: Suven Pharmaceuticals Limited, Hyderabad (IN)

(72) Inventors: Mohana Rao Manam, Hyderabad (IN); Murali Krishna Prasad Vallabhaneni, Hyderabad (IN); Venkata Srikanth Meka, Hyderabad (IN); Venkateswaralu Jasti, Hyderabad (IN)

(21) Appl. No.: 18/018,018

(22) PCT Filed: Jul. 28, 2021
(86) PCT No.: PCT/IB2021/056855

§ 371 Date: Jan. 25, 2023

(30) Foreign Application Priority Data
Jul. 28, 2020 (IN) 202041032260

(51) Int. Cl. A61K 31/40; A61K 9/00; A61K 47/12; A61K 47/26; A61K 47/10; A61K 47/38

(52) U.S. Cl. CPC A61K 31/40; A61K 9/006; A61K 47/12; A61K 47/26; A61K 47/10; A61K 47/38

(57) ABSTRACT

The present invention relates to novel oral film composition comprising anticholinergic agent as active ingredient and pharmaceutically acceptable excipients. The present invention specifically relates to novel oral film composition comprising Glycopyrronium salts as active ingredient and pharmaceutically acceptable excipients. The present invention more specifically relates novel oral film composition comprising Glycopyrronium salts, saliva stimulating agents, film-forming agents, buffering agents, plasticizers and other pharmaceutically acceptable excipients. The present invention more specifically relates to process for the preparation of Glycopyrronium salts oral film comprising the steps of dissolving/mixing, stirring, drying and cutting into film.

### FIELD OF INVENTION

The present invention relates to novel oral film composition comprising anticholinergic agent as active ingredient and pharmaceutically acceptable excipients.

The present invention specifically relates to novel oral film composition comprising Glycopyrronium salts as active ingredient and pharmaceutically acceptable excipients.

The present invention more specifically relates novel oral film composition comprising Glycopyrronium salts, saliva stimulating agents, film-forming agents, buffering agents, plasticizers and other pharmaceutically acceptable excipients.

The present invention more specifically relates to process for the preparation of Glycopyrronium salts oral film comprising the steps of dissolving/mixing, stirring, drying and cutting into film.

### BACKGROUND OF INVENTION

Glycopyrronium is a synthetic quaternary ammonium anticholinergic agent which inhibits the muscarinic actions of acetylcholine on autonomic nerve endings, decreasing respiratory and gastrointestinal secretions and intestinal motility. Glycopyrronium has broad activity against muscarinic acetylcholine receptors, but its highly polar quaternary ammonium group makes it less likely to cross lipid membranes such as the blood brain barrier, which is believed to decrease the potential for central nervous system effects. Glycopyrronium directly reduces the rate of salivation by preventing the stimulation of these receptors. Glycopyrronium indicated to reduce chronic severe drooling in patients aged 3 to 16 years with neurologic conditions associated with problem drooling (e.g., cerebral palsy). Glycopyrronium salts (Glycopyrronium bromide) has been used largely for gastrointestinal conditions including peptic ulcer disease, gastrointestinal spasm and irritable colon.

Glycopyrronium decreases acid secretion in the stomach. Hence it can be used for treating ulcers in the stomach and small intestine, in combination with other medications. In anesthesia, Glycopyrronium salts injection serves as a preoperative antimuscarinic operation that reduces salivary, tracheobronchial, and pharyngeal secretions, as well as decreases the acidity of gastric secretions blocks cardiac vagal inhibitory reflexes during intubation.

Glycopyrronium is chemically described as, 3-[(2-cyclopentyl-2-hydroxy-2-phenylacetyl)oxy]-1,1-dimethyl-pyrrolidinium. Glycopyrronium base has a chemical formula of C₁₉H₂₈NO₃ and a molecular mass of 318.4 g/mol.

Glycopyrronium is available in different salt forms such as bromide, acetate, benzoate, edisylate, oxalate, hydrogen sulfate and tosylate.

The general structural formula of Glycopyrronium salts is given below:

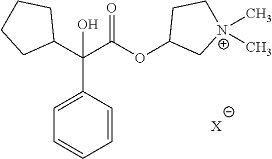

One such salt form which is currently marketed is Glycopyrronium salts (Glycopyrronium bromide), chemically known as pyrrolidinium, 3-[(2-cyclopentyl-2-hydroxy-2-phenylacetyl)oxy]-1,1-dimethyl-,bromide. Glycopyrronium salts has a chemical formula of C₁₉H₂₈BrNO₃ and a molecular mass of 398.33 g/mol.

Glycopyrronium is available in the market in the form of injection, tablets, solution and inhalation.

Sialorrhea (drooling or excessive salivation) is a common problem in neurologically impaired children (i.e., those with mental retardation or cerebral palsy) and in adults who have Parkinson's disease or have had a stroke. Glycopyrronium is reported to be used as effective initial method of treating hyperhidrosis/reducing Sweat and salivary gland secretions (Refer: The American Journal of Digestive Diseases, 1967, 12(5), 439-448; Diabetology, 1997, 40, 299-301; Korean J Pain. 2012 January; 25(1): 28-32).

U.S. Pat. No. 5,948,430 A discloses rapidly dissolving film which can be adhered to the oral cavity thereby releasing a pharmaceutically or cosmetically active agent, said film comprising water-soluble polymers, one or more polyalcohols, and one or more pharmaceutically or cosmetically active ingredients. Optionally, the formulation may contain a combination of certain plasticizers or surfactants, colorants, sweetening agent, flavors, flavor enhancers or other excipients commonly used to modify the taste of formulations intended for application to the oral cavity. It discloses antispasmodic agents as pharmaceutically active ingredients which covers Glycopyrronium salts generically.

U.S. Pat. No. 7,399,482 B1 discloses thin film medication device for therapeutic use, said device comprising: at least one support sheet; and a plurality of application strips attached to said support sheet, said strip comprising: a polymer base; and a therapeutic agent admixed with said polymer base. It discloses antispasmodic agents as pharmaceutically active ingredients which covers Glycopyrronium salts generically.

U.S. Pat. No. 7,579,019 B2 discloses pharmaceutical delivery device for application of a pharmaceutical to mucosal surfaces. The device comprises an adhesive layer and a non-adhesive backing layer, and the pharmaceutical may be provided in either or both layers. Upon application, the device adheres to the mucosal surface, providing localized drug delivery and protection to the treatment site.

U.S. Pat. No. 8,603,514 B2 discloses rapid dissolve thin film drug delivery compositions for the oral administration of active components. The active components are provided as taste-masked or controlled-release coated particles uniformly distributed throughout the film composition. The compositions may be formed by wet casting methods, where the film is cast and controllably dried, or alternatively by an extrusion method.

U.S. Pat. No. 8,765,167 B2 discloses an edible film for delivery of an active including: an edible, water-soluble polymer; at least one anti-tacking agent selected from the group consisting of lubricants, antiadherants, glidants and combinations thereof; and an active component selected from the group consisting of cosmetic agents, pharmaceutical agents, vitamins, bioactive agents and combinations thereof, wherein the film is self-supporting.

U.S. Pat. No. 9,125,836 B2 discloses film preparation having both rapid solubility and flexibility at the same time. The film preparation is characterized by comprising: an active-ingredient-containing layer comprising an active ingredient, a water-soluble polymer selected from the group consisting of methyl cellulose, hydroxypropyl cellulose, and hydroxypropyl methyl cellulose, and a disintegrating agent; and an active-ingredient-free layer comprising methyl cellulose and/or hydroxypropyl methyl cellulose. It discloses antispasmodic agents as pharmaceutically active ingredients which covers Glycopyrronium salts generically.

U.S. Pat. No. 9,717,682 B2 discloses solid oral film dosage forms for the buccal and/or sublingual delivery of pharmaceutical, nutraceutical or cosmetic ingredients are endowed with instant hydration potential and complete dissolution potentially enabling the active ingredient to become immediately available for enhanced buccal and/or sublingual absorption and/or reduced absorption through the gastrointestinal route. The improved delivery systems for solubilizing and stabilizing pharmaceutically active ingredients exhibit enhanced stability by the use of a combination of crystallization inhibitors, which together can maintain the active ingredient in a desired plurality of particles in an effective size range within a polymeric film matrix.

US Publication No. 2007/0207192 A1 discloses a mucoadhesive delivery system which comprises: an at least partially water-soluble bioadhesive layer comprising at least one bioadhesive polymer or a combination of at least one bioadhesive polymer and at least one first film-forming, water-soluble polymer; an at least partially water-soluble non-adhesive backing layer comprising at least one second water-soluble, film-forming polymer; at least one pharmaceutical agent, and; a mucosal penetration enhancing agent; wherein the system is mucoadhesive, is flexible and is biodegradable.

WO Publication No. 2001/008681 A1 discloses methods and compositions for the treatment of various conditions that interfere with patients' lifestyles, lead to social isolation, and involve pain and debilitation. Methods are provided for the treatment of conditions such as Frey's Syndrome, gustatory sweating, hyperhidrosis, sialorrhea, myasthenia gravis and Meniere's disease. Preferably, these conditions are treated with methods of administration of compositions of Glycopyrronium salts. The present invention comprises methods of administration of Glycopyrronium salts compositions comprising injectable and non-invasive routes for drug delivery, including but not limited to, the oral, nasal, pulmonary, rectal, buccal, vaginal, transdermal and ocular routes.

None of the prior art references disclose Glycopyrronium Oral film compositions specifically. The inventors of the present invention provide composition of oral film comprising Glycopyrronium salts, saliva stimulating agent, film forming agent, buffering agent, sweetening agent, plasticizer, flavouring agents, solvents and other pharmaceutically acceptable excipients which is easy to manufacture, pack, carrying and administer. The inventors of present invention also provide process for the preparation of Glycopyrronium oral film comprising the steps of dissolving/mixing, stirring, drying and cutting into film.

### OBJECTIVE OF INVENTION

The main objective of the present invention is to provide anticholinergic agent oral film composition.

Another objective of the present invention is to provide novel oral film composition comprising Glycopyrronium salts as active ingredient and pharmaceutically acceptable excipients.

Still another objective of the present invention is to provide novel oral film composition comprising Glycopyrronium salts, saliva stimulating agents, film-forming agent, buffering agents, plasticizers and other pharmaceutically acceptable excipients.

Still another objective of the present invention is to provide process for the preparation of Glycopyrronium salts oral film comprising the steps of dissolving/mixing, stirring, drying and cutting into film.

### SUMMARY OF INVENTION

Accordingly, the present invention provides a composition of anticholinergic agent oral film.

One embodiment of the present invention provides a composition of Glycopyrronium salts oral film.

One embodiment of the present invention provides a composition of Glycopyrronium salts oral film for use in the treatment of hyperhidrosis, Sialorrhea (Excessive Drooling). Specifically, Glycopyrronium salts oral film can be used to reduce chronic severe drooling in patients aged 3 to 16 years with neurologic conditions associated with problem drooling (e.g., cerebral palsy). Glycopyrronium salts oral film can also use as adjunctive therapy in the treatment of peptic ulcer when rapid anticholinergic effect is desired or when oral medication is not tolerated.

Another embodiment of the present invention provides a novel oral film composition comprising Glycopyrronium salts and pharmaceutically acceptable excipients.

Another embodiment of the present invention provides a novel oral film composition comprising Glycopyrronium salts, saliva stimulating agents, film-forming agent, buffering agents, plasticizer and other pharmaceutically acceptable excipients.

Another embodiment of the present invention provides a novel oral film composition comprising Glycopyrronium salts and one or more pharmaceutically acceptable excipients which are selected from saliva stimulating agents, film-forming agents, buffering agents, plasticizers, permeation enhancers, preservatives, humectants, antioxidants, superdisintegrants, taste modifying agents, colouring agents, stabilizers, thickening agents, emulsifiers, sweetening agents and flavouring agents.

Another embodiment of the present invention provides a novel Glycopyrronium salts oral film composition comprising:
0.05% to 5% (w/w) of Glycopyrronium salts,
0.1% to 10% (w/w) of saliva stimulating agents,
0.5% to 60% (w/w) of film-forming agents,
0.05% to 10% (w/w) of buffering agents,
0.1% to 25% (w/w) of plasticizers, and
0.5% to 90% (w/w) of other pharmaceutically acceptable excipients.

Yet another embodiment of the present invention provides a process for the preparation of Glycopyrronium salts oral film comprising the steps of:
(a) dissolving saliva stimulating agent, buffering agent and Glycopyrronium salts in purified water,
(b) adding additional film forming agents/polymers to the solution of step (b) under continuous stirring to form clear solution,
(c) adding plasticizers and other pharmaceutical acceptable excipients to the obtained solution of step (b) under continuous stirring to form clear solution/uniform dispersion,
(d) pouring the obtained solution of step (c) on inert support in required thickness, and
(e) drying the film at the temperature of 50-60° C. and cutting into required sizes/shapes.

Yet another embodiment of the present invention provides process for the preparation of Glycopyrronium salts oral film comprising the steps of:
(a) mixing stabilizer, plasticizer, film forming agent, taste masking agent in purified water under continuous stirring,
(b) adding solvent and additional taste masking agent to solution of step (a),
(c) dissolving saliva stimulating agent and buffering agent in purified water separately,
(d) adding Glycopyrronium salts to buffer solution of step (c) under continuous stirring,
(e) adding Glycopyrronium salts solution to the solution of step (b), and
(f) placing obtained mixture under vacuum, casting as a uniform layer onto an inert support, drying in an oven and cutting into required sizes/shapes.

Yet another embodiment of the present invention provides process for the preparation of Glycopyrronium salts oral film comprising the steps of:
(a) adding thickening agent in purified water under continuous stirring to form clear liquid,
(b) adding buffering agent, antioxidant, plasticizer, film forming agent under continuous stirring,
(c) dissolving saliva stimulating agent and additional buffering agent in purified water separately,
(d) adding Glycopyrronium salts to buffer and dissolving it under continuous stirring,
(e) adding Glycopyrronium salts solution to the solution of step (b),
(f) adding solvent to the solution of step (e) under continuous stirring to form uniform mixture, and
(g) placing obtained mixture under vacuum, casting as a uniform layer onto an inert support, drying in an oven and cutting into required sizes/shapes.

### DETAILED DESCRIPTION OF THE INVENTION

The term “comprising”, which is synonymous with “including”, “containing”, or “characterized by” here is defined as being inclusive or open-ended, and does not exclude additional, unrecited elements or method steps, unless the context clearly requires otherwise.

The present invention provides novel oral film composition comprising Glycopyrronium salts as active ingredient, pharmaceutically acceptable excipients and its process of preparation.

The present invention provides novel oral film composition comprising Glycopyrronium as active ingredient and one or more pharmaceutically acceptable excipients which are selected from saliva stimulating agents, permeation enhancer, film-forming agent, plasticizer, humectant, antioxidant, superdisintegrants, sweetener, taste modifying agent, buffering agent, colouring agent, stabiliser, thickening agent, emulsifier, preservative, flavouring agent as pharmaceutically acceptable excipients.

The term “active ingredient” of the present invention includes anticholinergic agent. Preferably used anticholinergic agent is Glycopyrronium salts such as bromide, acetate, benzoate, edisylate, oxalate, hydrogen sulfate and tosylate.

The concentration of Glycopyrronium base or its salts used in the present invention is in the range of 0.05% to 5% (w/w), more preferably 0.1 to 3% (w/w) of the total weight of the composition.

Saliva stimulating agent used alone or in combination in the compositions of the present invention include, but are not limited to citric acid anhydrous, malic acid, lactic acid, tartaric acid and ascorbic acid. Preferably used saliva stimulating agent is citric acid anhydrous.

The concentration of saliva stimulating agent in the Glycopyrronium salts oral film is in the range of 0.1% to 10% (w/w), more preferably 0.15% to 2% (w/w) of the total weight of the composition.

Film-forming agents used alone or in combination in the compositions of the present invention include, but are not limited to hydroxypropyl methylcellulose grades, hydroxy propyl cellulose, Sodium carboxy methyl cellulose, polyvinyl alcohol, polyvinyl acetate, hydroxyethyl cellulose (HEC), methylcellulose (MC), ethylcellulose (EC), polyvinyl pyrrolidone (copovidone), polyethyleneoxide, carrageenan, gelatin, dextrin, polyethylene oxide, Starch, Pectin, Sodium alginate, guar gum, xanthan gum, carrageenan, Glycerol monooleate, maltodextrin, Tragacanth gum, sodium citrate dihydrate, polyvinyl alcohol-polyethylene glycol co-polymers, carboxylic acid containing polymers such as acrylic acid, methacrylic acid, esterified poly acrylic acid polymers, such as polyacrylic acid polymers lightly crosslinked with a polyalkenylpolyethers; methacrylate polymers; maleic acid copolymers; methacrylic acid-ethyl acrylate copolymers, methacrylic acid and methacrylate based polymers, and a methylmethacrylate copolymer. Preferably used Film-forming agents include polyvinyl alcohol, hydroxypropyl methylcellulose, maltodextrin, HPMC grades like Methocel E50 and Methocel E5, polyethylene oxide or combinations thereof.

The concentration of film-forming agents in the Glycopyrronium salts oral film is in the range of 0.5% to 60% (w/w) of the total weight of the composition, more preferably 2% to 50% (w/w) of the total weight of the composition.

Buffering agents used alone or in combination in the compositions of the present invention include, but are not limited to citric acid, sodium carbonate, sodium citrate dihydrate, trisodium citrate dihydrate, ascorbic acid, acetic acid, tartaric acid, citric acid monohydrate, sodium bicarbonate, potassium carbonate, potassium bicarbonate, sodium phosphate dibasic, sodium phosphate tribasic, disodium phosphate dibasic, potassium citrate, potassium phosphate dibasic, potassium phosphate tribasic, tricalcium phosphate, calcium carbonate, calcium phosphate, carbonated calcium phosphate, magnesium carbonate, sodium hydroxide, magnesium hydroxide, potassium hydroxide, aluminium hydroxide, and combinations thereof. Preferably used buffering agents include sodium citrate dihydrate, sodium phosphate tribasic and disodium phosphate dibasic or combinations thereof.

The concentration of buffering agents in the Glycopyrronium salts oral film is in the range of 0.05% to 10% (w/w) of the total weight of the composition, more preferably 0.05% to 5% (w/w) of the total weight of the composition.

Plasticizers used alone or in combination in the compositions of the present invention include, but are not limited to polyalkylene oxides, such as polyethylene glycols, polypropylene glycols, polyethylene-propylene glycols, organic plasticizers with low molecular weights, such as glycerol, glycerol monoacetate, diacetate or triacetate, triacetin, polysorbate, cetyl alcohol, propylene glycol, sorbitol, sodium diethylsulfosuccinate, triethyl citrate, glycerine, tributyl citrate and the like. Preferably used plasticizers include polyethylene glycol 400, polysorbate 80, propylene glycol and glycerine or combinations thereof.

The concentration of plasticizers in the Glycopyrronium salts oral film is in the range of 0.1% to 25% (w/w) of the total weight of the composition, more preferably 1% to 20% (w/w) of the total weight of the composition.

Humectant used alone or in combination in the compositions of the present invention include, but are not limited to water soluble liquid polyols selected from the group comprising glycerine, glycerol, Sorbitol, Xylitol, butylene glycol, polyethylene glycol, urea, Sodium lactate, Sodium pyrrolidone carboxylic acid (PCA), hyaluronic acid (sodium salt), carrageenan and agarose, propylene glycol, hexylene glycol, butylene glycol, pentylene glycol, dipropylene glycol, and mixtures thereof. Preferably used humectant is glycerol.

The concentration of humectant in the Glycopyrronium salts oral film is in the range of 0.5% to 20% (w/w), more preferably 1% to 15% (w/w) of the total weight of the composition.

Antioxidant used alone or in combination in the compositions of the present invention include, but are not limited to disodium EDTA, beta carotene, alpha hydroxy acids such as glycolic acid, citric acid, lactic acid, malic acid, mandelic acid, ascorbic acid, alpha-hydroxybutyric acid, alpha-hydroxyisobutyric acid, alpha-hydroxyisocaprolc acid, atrrolactic acid, alpha-hydroxyisovaleric acid, ethyl pyruvate, galacturonic acid, glucoheptonic acid, glucoheptono 1,4-lactone, gluconic acid, gluconolactone, glucuronic acid, glucuronolactone, glycolic acid, isopropyl pyruvate, methyl pyruvate, mucic acid, pyruvic acid, saccharic acid, saccharic acid 1,4-lactone, tartaric acid and tartronic acid; beta hydroxy acids such as beta-hydroxybutyric acid, beta-phenyl-lactic acid and beta-phenylpyruvic acid. Preferably used antioxidant is disodium EDTA.

The concentration of antioxidant in the Glycopyrronium salts oral film is in the range of 0.1% to 10% (w/w), more preferably 0.1% to 1% (w/w) of the total weight of the composition.

Superdisintegrant used alone or in combination in the compositions of the present invention include, but are not limited to cross-linked polyvinylpyrrolidone, cross-linked sodium carboxymethylcellulose, carboxymethyl starch, sodium carboxymethyl starch, potassium methacrylate-divinylbenzene copolymer, polyvinyl alcohols, amylose, cross-linked amylose, starch derivatives, microcrystalline cellulose and cellulose derivatives, alpha-, beta- and gamma-cyclodextrin and dextrin derivatives; an acrylic acid polymer such as cross-linked polymer available under the tradename Carbopol®; a vinyl pyrrolidone polymer such as crosslinked polyvinylpyrrolidone or crospovidone; copolymers of vinyl pyrrolidone and vinyl acetate; or mixtures thereof. Preferably used superdisintegrant includes starch.

The concentration of superdisintegrant in the Glycopyrronium salts oral film is in the range of 1% to 25% (w/w), more preferably 5% to 20% (w/w) of the total weight of the composition.

Sweeteners used alone or in combination in the compositions of the present invention include, but are not limited tonatural and artificial sweeteners. Non-limiting examples of suitable sweeteners include, e.g: water-soluble sweetening agents such as monosaccharides, disaccharides and polysaccharides such as xylose, ribose, glucose (dextrose), mannose, galactose, fructose (levulose), sucrose (sugar), high fructose corn syrup, maltose, invert sugar (a mixture of fructose and glucose derived from sucrose), partially hydrolyzed starch, corn syrup solids, and dihydrochalcones; water-soluble artificial sweeteners such as the soluble saccharin salts, i.e., sodium or calcium saccharin salts, cyclamate salts, the sodium, ammonium or calcium salt of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide (acesulfame-K), the free acid form of saccharin and the like; dipeptide based sweeteners, such as L-aspartic acid derived sweeteners, such as L-aspartyl-L-phenylalanine methyl ester (aspartame), L-alpha-aspartyl-N-(2,2,4,4-tetramethyl-3-thietanyl)-D-alaninamide hydrate, methyl esters of L-aspartyl-L-phenylglycerin and L-aspartyl-L-2,5,dihydrophenylglycine, L-aspartyl-2,5-dihydro-L-phenylalanine, L-aspartyl-L-(1-cyclohexyen)-alanine, and the like; water-soluble sweeteners derived from naturally occurring water-soluble sweeteners, such as a chlorinated derivatives of ordinary sugar (sucrose), known, for example, as sucralose; and protein based sweeteners such as thaurnatoccous danielli (Thaurnatin I and II), naturally occurring high intensity sweeteners, such as Lo Han Kuo, stevia, steviosides, monellin, Neotame, Alitame, Sucrose, Fructose, Sorbitol, Mannitol, Aspartame, Saccharin Sodium, Glucose, Dextrose, Isomaltose, Acesulfame Potassium, Sorbitol, Ribose, Mannose, Galactose, Maltose, Partially Hydrolyzed Starch, Corn Syrup, Xylitol, Mannitol, Saccharin Salts, Cyclamate Salts, Thaumatin I & II, Natural and Artificial bitter masker, Advantame, Tagatose, Stevia, glycyrrhizin, sodium cyclamate and honey. Preferably used sweeteners include acesulfame potassium, aspartame and sorbitol or combinations thereof.

The concentration of sweeteners in the Glycopyrronium salts oral film is in the range of 0.05% to 40% (w/w) of the total weight of the composition, more preferably 0.1% to 20% (w/w) of the total weight of the composition.

Taste modifying agents used alone or in combination in the compositions of the present invention include, but are not limited to flavoring agents, sweetening agents and taste masking agents and are exemplified by: the essential oils or water soluble extracts of menthol, wintergreen, peppermint, sweet mint, spearmint, natural and artificial vanilla, cherry, chocolate, fudge, butterscotch, cinnamon, clove, lemon, orange, raspberry, rose, spice, violet, herbal, fruit, strawberry, grape, pineapple, peach, kiwi, papaya, mango, coconut, apple, coffee, plum, watermelon, nuts, durean, green tea, grapefruit, banana, butter, cream custard, camomile, sugar, dextrose, lactose, mannitol, sucrose, xylitol, maltitol, acesulfame potassium, talin, glycyrrhizin, sucralose, aspartame, saccharin, sodium saccharin, sodium cyclamate and honey. Preferably used taste modifying agents include menthol and sucralose.

The concentration of taste modifying agents in the Glycopyrronium salts oral film is in the range of 0.05% to 40% (w/w) of the total weight of the composition, more preferably 0.1% to 20% (w/w) of the total weight of the composition.

Taste masking agents also can be used in the compositions of the present invention include, but are not limited to polymers such as polymers, oils, or waxes. Polymers can be used amino alkyl methacrylate copolymers (Eudragit E-100), ethylcellulose, HPMC, HPC, polyvinyl alcohol, and polyvinyl acetate.

Colouring agent used alone or in combination in the compositions of the present invention include, but are not limited to titanium dioxide, riboflavin, beta carotene, anthocyanidin, fuchsin, indigo-blue fuchsin, orange Yellow S, quinoline yellow, indigo-blue acid blue (indigotine lake), light blue and sunset yellow. Preferably used colouring agent is titanium dioxide.

The concentration of colouring agent in the Glycopyrronium salts oral film is in the range of 0.1% to 5% (w/w), more preferably 0.5% to 1% (w/w) of the total weight of the composition.

Stabiliser used alone or in combination in the compositions of the present invention include, but are not limited to D-α-tocopheryl polyethylene glycol succinate (TPGS), butylated hydroxyanisole (BHA), butylated hydroxytoluene (BHT), t-butyl hydroquinone, calcium ascorbate, gallic acid, hydroquinone, maltol, octylgallate, sodium bisulfite, sodium metabisulfite, tocopherol and derivatives thereof, citric acid, tartaric acid, and ascorbic acid. Preferably used stabiliser is sodium metabisulfite.

The concentration of stabiliser in the Glycopyrronium salts oral film is in the range of 0.1% to 5.0% (w/w), more preferably 0.1% to 2.0% (w/w) of the total weight of the composition.

Thickening agent used alone or in combination in the compositions of the present invention include, but are not limited to methyl cellulose, xanthan gum, sodium carboxymethyl cellulose, hydroxypropyl cellulose, carbomer, polyvinyl alcohol, alginates, acacia, chitosans and combinations thereof. Preferably used thickening agent is Sodium CMC 7L2P.

The concentration of thickening agent in the Glycopyrronium salts oral film is in the range of 5% to 10% (w/w), more preferably 6% to 9% (w/w) of the total weight of the composition.

Emulsifier used alone or in combination in the compositions of the present invention include, but are not limited to castor oil derivatives, cetyl and palmityl alcohol, ethanol, hydrogenated vegetable oils, polyvinyl alcohol, simethicone, sorbitan ester, glyceryl monostearate, polyoxyethylene alkyl ethers, polyoxyethylene stearates, poloxamer, polyoxyethylene lauryl ether and polyoxyethylene sorbitan fatty acid esters. Preferably used emulsifier is polyoxyethylene lauryl ether.

The concentration of emulsifier in the Glycopyrronium salts oral film is in the range of 0.1% to 5% (w/w), more preferably 1% to 3% (w/w) of the total weight of the composition.

Flavouring agents used alone or in combination in the compositions of the present invention include, but are not limited to natural and artificial flavors. These flavorings may be chosen from synthetic flavor oils and flavoring aromatics, and/or oils, oleo resins and extracts derived from plants, leaves, flowers, fruits and so forth, and combinations thereof. Non-limiting flavor oils include: spearmint oil, cinnamon oil, peppermint oil, clove oil, bay oil, thyme oil, cedar leaf oil, oil of nutmeg, oil of sage, and oil of bitter almonds. Also useful are artificial, natural or synthetic fruit flavors such as vanilla, chocolate, coffee, cocoa and citrus oil, including lemon, orange, grape, lime and grapefruit, and fruit essences including apple, pear, peach, strawberry, raspberry, cherry, plum, pineapple, apricot and the like. These flavorings can be used individually or in combination. Commonly used flavors include mints such as peppermint, artificial vanilla, cinnamon derivatives, and various fruit flavors, whether employed individually or in combination. Flavorings such as aldehydes and esters including cinnamylacetate, cinnamaldehyde, citral, diethylacetal, dihydrocarvyl acetate, eugenyl formate, p-methylanisole, and the like may also be used. Further examples of aldehyde flavorings include, but are not limited to acetaldehyde (apple); benzaldehyde (cherry, almond); cinnamaldehyde (cinnamon); citral, i.e., alpha citral (lemon, lime); neral, i.e. beta citral (lemon, lime); decanal (orange, lemon); ethyl vanillin (vanilla, cream); heliotropine, i.e., piperonal (vanilla, cream); vanillin (vanilla, cream); alpha-amyl cinnamaldehyde (spicy fruity flavors); butyraldehyde (butter, cheese); valeraldehyde (butter, cheese); citronellal (modifies, many types); decanal (citrus fruits); aldehyde C-8 (citrus fruits); aldehyde C-9 (citrus fruits); aldehyde C-12 (citrus fruits); 2-ethyl butyraldehyde (berry fruits); hexenal, i.e. trans-2 (berry fruits); tolyl aldehyde (cherry, almond); veratraldehyde (vanilla); 12,6-dimethyl-5-heptenal, i.e. melonal (melon); 2 dimethyloctanal (greenfruit); and 2-dodecenal (citrus, mandarin); cherry; anis flavour; grape; mixtures thereof; and the like. Preferably used flavouring agents include cherry flavour, anis flavour, peppermint flavour and lemon mint flavor.

The concentration of flavouring agents in the Glycopyrronium salts oral film is in the range of 0.1% to 15% (w/w) of the total weight of the composition. Permeation enhancer may used alone or in combination in the compositions of the present invention include, but are not limited to β-cyclodextrin, isopropyl palmitate, isopropyl myristate, isopropyl stearate, propylene glycol, octyl stearate, tridecyl neopentanoate, benzyl alcohol, linoleic acid, alpha-linolenic, oleic acid, cod-liver-oil, methanol, menthol derivatives, squalene, glycerol derivatives, urea, sodium taurocholate or a combination thereof. Preferably used permeation enhancer is β-cyclodextrin.

The concentration of permeation enhancer in the Glycopyrronium salts oral film is in the range of 1% to 25% (w/w), more preferably 10% to 23% (w/w) of the total weight of the composition.

Solvents used alone or in combination in the compositions of the present invention include, but are not limited to water, methanol, ethanol, propanol, or low alkyl alcohols such as isopropyl alcohol, or acetone. Other suitable solvents may comprise dimethyl acetamide, N-methyl-2-pyrrolidone, dimethyl sulfoxide, ethoxydiglycoli, propylene glycol, polyethylene glycol. Preferably used solvents include ethanol, purified water, isopropyl alcohol and acetone.

The concentration of solvents in the Glycopyrronium salts oral film is in the range of 5% to 90% (w/w) of the total weight of the composition, more preferably 10% to 85% (w/w) of the total weight of the composition.

Preservative used alone or in combination in the compositions of the present invention include, but are not limited to Ethylparaben, Propylparaben, Methylparaben, potassium sorbate, Butylated Hydroxy Toluene, Sodium benzoate, benzoic acid, boric acid, sorbic acid or their salts thereof, benzyl alcohol, benzalkonium chloride, para hydroxybenzoic acids and their alkyl esters, or the mixtures thereof. Preferable preservatives can be methyl paraben, Ethylparaben and propyl paraben.

The concentration of preservatives in the Glycopyrronium salts oral film is in the range of 0.01% to 10% (w/w), more preferably 0.12% to 2% (w/w) of the total weight of the composition.

Another embodiment of the present invention provides Glycopyrronium salts oral film comprising:
0.05% to 5% (w/w) of Glycopyrronium salts,
0.1% to 10% (w/w) of citric acid anhydrous,
0.5% to 60% (w/w) of film-forming agents selected from polyvinyl alcohol, hydroxypropyl methylcellulose, maltodextrin, HPMC grades like Methocel E50 and Methocel E5, polyethylene oxide or combinations thereof,
0.05% to 10% (w/w) of buffering agents selected from sodium citrate dihydrate, sodium phosphate tribasic and disodium phosphate dibasic or combinations thereof,
0.1% to 25% (w/w) of plasticizers selected from, polyethylene glycol 400, polysorbate 80, propylene glycol and glycerine or combinations thereof, and
0.5% to 90% (w/w) of other pharmaceutically acceptable excipients.

The present invention is illustrated in detail but not limiting to, the following examples. It will be apparent to those skilled in the art that many modifications, both to materials and methods, may be practiced without departing from the scope of the invention.

### Example 1

| | | Concentration |
|---|---|---|
| S. No | Ingredients | (% w/w) |
| 1. | Glycopyrronium salts | 0.50 |
| 2. | Citric acid anhydrous | 0.15 |
| 3. | Sodium citrate dihydrate | 0.06 |
| 4. | Polyvinyl alcohol | 18.76 |
| 5. | Polyethylene glycol 400 | 5.10 |
| 6. | Glycerol | 1.70 |
| 7. | Starch | 8.50 |
| 8. | Menthol | 0.85 |
| 9. | Sucralose | 0.17 |
| 10. | Ethanol | 25.35 |
| 11. | Purified water | 38.86 |

### Manufacturing Process:

1. Preparation of Drug solution:
   Citric acid anhydrous and sodium citrate dihydrate were dissolved in Purified water. Glycopyrronium was added under continuous stirring until forms a clear solution.
2. Addition of Film forming agents/Polymers:

Polyvinyl alcohol was added slowly to the drug solution under continuous stirring until clear solution formed.
3. Addition of Plasticizers
   Polyethylene glycol 400 and Glycerol were added under continuous stirring until clear solution formed.
4. Addition of bulking agent:
   Starch was added to the solution under continuous stirring.
5. Addition of Sweetening Agent:
   Sucralose was added under continuous stirring.
7. Addition of Flavouring agent:
   Menthol was added to the solution under continuous stirring.
8. Addition of Ethanol.
   Ethanol was added to the dispersion under continuous stirring.
9. Solvent casting:
   The solution was poured on Norfilm in required thickness by using Equipment: Mathis Lab coater (Model No: LTE-S, Mfr.: Werner Mathis AG, Switzerland). After uniform distribution of the dispersion, the film was dried at the temperature of 50-60° C. The film can be cut in required sizes/shapes.

### Example 2

| | | Concentration |
|---|---|---|
| S. No | Ingredients | (% w/w) |
| 1. | Glycopyrronium salts | 1.00 |
| 2. | Citric acid anhydrous | 0.15 |
| 3 | Sodium citrate dihydrate | 0.06 |
| 4. | Polyvinyl alcohol | 22.00 |
| 5. | Polyethylene glycol | 11.88 |
| 6. | Glycerol | 3.96 |
| 7. | Starch | 19.80 |
| 8 | Acesulfame Potassium | 0.40 |
| 9. | Titanium dioxide | 0.50 |
| 10. | Menthol | 1.90 |
| 11. | Polysorbate 80 | 1.00 |
| 12. | Ethanol | 18.5 |
| 13. | Purified water | 18.5 |

### Manufacturing Process:

Citric acid anhydrous and sodium citrate dihydrate were dissolved in Purified water. Glycopyrronium was added under continuous stirring until clear solution formed. Polyvinyl alcohol was added slowly to the drug solution under continuous stirring. Polyethylene glycol, Polysorbate 80 and Glycerol were added under continuous stirring. Starch was added to the solution under continuous stirring until forms a uniform dispersion. Acesulfame potassium was added under continuous stirring. Titanium dioxide was added to the above mixture with stirring for 15 min. Menthol was added to the solution under continuous stirring and mixed for 10 min. Ethanol added slowly under continuous stirring for 5 min.
The solution was poured on Norfilm in required thickness by using Equipment: Mathis Lab coater (Model No: LTE-S, Mfr.: Werner Mathis AG, Switzerland). After uniform distribution of the dispersion, the film was dried at the temperature of 50-60° C. The film can be cut in required sizes/shapes.

### Example 3

| | | Concentration |
|---|---|---|
| S. No | Ingredients | (% w/w) |
| 1. | Glycopyrronium salts | 1.50 |
| 2. | Citric acid anhydrous | 0.20 |
| 3. | Sodium citrate dihydrate | 0.15 |
| 4. | Hydroxypropyl methylcellulose | 19.50 |
| 5. | Polyvinyl alcohol | 10.00 |
| 6. | Sucralose | 1.05 |
| 7. | Menthol | 1.00 |
| 8. | Cherry flavour | 0.50 |
| 9 | Ethanol | 34.10 |
| 10. | Purified water | 32.00 |

### Manufacturing Process:

Citric acid anhydrous, sodium citrate dihydrate and Glycopyrronium salts were dissolved in Purified water. Hydroxypropylmethyl cellulose, polyvinyl alcohol and menthol were dissolved in purified water in a separate container under mixing. Polymer mixture was added to the drug solution under continuous stirring and mixed for 10-15 min. Sucralose added to the mixture and mixed for 10 min. Cherry flavour and ethanol were added to the mixture and mixed for 10 min.
The solution was poured on Norfilm in required thickness by using Equipment: Mathis Lab coater (Model No: LTE-S, Mfr.: Werner Mathis AG, Switzerland). After uniform distribution of the dispersion, the film was dried at the temperature of 50-60° C. The film can be cut in required sizes/shapes.

### Example 4

| | | Concentration |
|---|---|---|
| S. No | Ingredients | (% w/w) |
| 1. | Glycopyrronium salts | 1.50 |
| 2. | Citric acid anhydrous | 0.15 |
| 3 | Sodium citrate dihydrate | 0.06 |
| 4. | Polyethylene oxide | 47.36 |
| 5. | Polysorbate 80 | 1.23 |
| 6. | Glycerol | 14.73 |
| 7. | Titanium dioxide | 0.61 |
| 8 | Acesulfame Potassium | 1.84 |
| 9. | Anis flavour | 4.71 |
| 10. | Ethanol | 14.00 |
| 11. | Purified water | 14.00 |

### Manufacturing Process:

Citric acid anhydrous, sodium citrate dihydrate and Glycopyrronium were dissolved in Purified water. Polyethylene oxide dissolved in a mixture of water and ethanol. Polymer solution was added to the drug solution under continuous stirring and mixed for 15 min. Polysorbate, Glycerol and Titanium dioxide were added to the mixture under continuous stirring and mixed for 15 min. Acesulfame potassium was added to the mixture and mixed for 5 min. Anis flavour was added to the mixture and mixed for 5 min.
The solution was poured on Norfilm in required thickness by using Equipment: Mathis Lab coater (Model No: LTE-S, Mfr.: Werner Mathis AG, Switzerland). After uniform distribution of the dispersion, the film was dried at the temperature of 50-60° C. The film can be cut in required sizes/shapes.

### Example 5

| | | Concentration |
|---|---|---|
| S. No | Ingredients | (% w/w) |
| 1 | Glycopyrronium salts | 1.00 |
| 2. | Sodium citrate dihydrate | 0.08 |
| 3. | β-Cyclodextrin | 22.00 |
| 4. | Polyvinyl alcohol | 27.22 |
| 5. | Polyethylene Glycol 1000 | 7.58 |
| 6. | Propylene glycol | 4.74 |
| 7. | Ethyl paraben | 0.12 |
| 8. | Propyl paraben | 0.20 |
| 9. | Citric acid anhydrous | 0.95 |
| 10. | Acesulfame Potassium | 1.36 |
| 11. | Anis Flavour | 1.25 |
| 12. | Peppermint flavour | 3.50 |
| 13. | Isopropyl alcohol | 15.00 |
| 14. | Purified water | 15.00 |

### Manufacturing Process:

Citric acid anhydrous, sodium citrate dihydrate and Glycopyrronium were dissolved in purified water. Polyvinyl alcohol was added to the drug solution and mixed for 15 min. β Cyclodextrin, Polyethylene Glycol was dissolved in water under continuous stirring. This mixture was added to the drug solution and mixed for 15 min. Ethyl paraben and propyl paraben were dissolved in propylene glycol. This mixture was added to drug solution and mixed for 10 min. Isopropyl alcohol was added to the mixture and mixed for 10 min. Acesulfame potassium was added to the mixture and mixed for 5 min. Anis flavour and peppermint flavour were added to the mixture and mixed for 5 min.
The solution was poured on Norfilm in required thickness by using Equipment: Mathis Lab coater (Model No: LTE-S, Mfr.: Werner Mathis AG, Switzerland). After uniform distribution of the dispersion, the film was dried at the temperature of 50-60° C. The film can be cut in required sizes/shapes.

### Example 6

| | | Concentration |
|---|---|---|
| S. No | Ingredients | (% w/w) |
| 1. | Glycopyrronium salts | 0.10 |
| 2. | Citric acid anhydrous | 0.15 |
| 3. | Sodium citrate dihydrate | 0.06 |
| 4. | Methocel E50 | 4.33 |
| 5. | Methocel E5 | 3.33 |
| 6. | Propylene glycol | 2.29 |
| 7. | Sodium metabisulfate | 0.12 |
| 8. | Menthol | 1.14 |
| 9. | Maltodextrin | 3.63 |
| 10. | Sucralose | 0.55 |
| 11. | Acetone | 39.30 |
| 12. | Water | 45.00 |

### Manufacturing Process:

Sodium metabisulfite, propylene glycol, maltodextrin and Sucralose were mixed with water and stirred the mixture. Acetone and menthol were added to this solution, and stirred the mixture. Separately, Citric acid and Sodium Citrate were dissolved in Purified water. Glycopyrronium was added to buffer and dissolved it under continuous stirring. Drug solution was added to polymer solution under stirring. Hydroxypropyl methylcellulose grades (Methocel E50 and Methocel E5) were added slowly understirring until a uniform, clear, viscous liquid was produced. The resulting mixture was placed under vacuum to eliminate air bubbles, cast as a uniform layer onto an inert Support, and dried in an oven at 50-70° C. The film can be cut in required sizes/shapes.

### Example 7

| | | Concentration |
|---|---|---|
| S. No | Ingredients | (% w/w) |
| 1 | Glycopyrronium | 0.2 |
| 2. | Citric acid anhydrous | 0.15 |
| 3. | Sodium citrate dihydrate | 0.06 |
| 4. | Sodium phosphate tribasic | 3.35 |
| 5. | Disodium phosphate dibasic | 0.55 |
| 6. | Sodium Carboxymethyl cellulose | 8.16 |
| 7. | Glycerin | 1.92 |
| 8. | Sodium metabisulfite | 0.23 |
| 9. | Disodium EDTA | 0.18 |
| 10. | Maltodextrin | 5.20 |
| 11. | Acetone | 10.00 |
| 12. | Water | 70.00 |

### Manufacturing Process:

Film was prepared by slowly adding sodium carboxymethyl cellulose to water under stirring until a uniform, clear, viscous liquid is produced. Sodium phosphate tribasic, disodium phosphate dibasic, Sodium metabisulfite, disodium EDTA, glycerin, and maltodextrin were added, and the mixture was stirred. Separately, Citric acid and Sodium Citrate were dissolved in Purified water. Glycopyrronium salts was added to buffer and dissolved it under continuous stirring. Drug solution was added to it with stirring. Acetone was added to this Solution, and the mixture was stirred, until a uniform, clear, viscous liquid was produced. The resulting mixture was placed under vacuum to eliminate air bubbles, cast as a uniform layer onto an inert Support, and dried in an oven. The film can be cut in required sizes/shapes.

### Example 8

| | | Concentration |
|---|---|---|
| S. No | Ingredients | (mg/film) |
| 1. | Glycopyrronium | 2.0 |
| 2. | Citric acid anhydrous | 1.5 |
| 3. | Sodium citrate dihydrate | 0.6 |
| 4. | Hydroxypropyl methylcellulose | 30.0 |
| 5. | Polysorbate 80 | 3.5 |
| 6. | Sorbitol | 15.0 |
| 7. | Glycerol | 6.0 |
| 8. | Polyoxyethylene Lauryl Ether | 2.0 |
| 9. | Lemon mint flavor | 25.0 |
| 10. | Aspartame | 3.0 |
| 11. | Menthol | 15.0 |
| 12. | Ethanol* | 250.0 |
| 13. | Water* | 250.0 |

*Evaporated during process.

### Manufacturing Process:

Citric acid anhydrous, sodium citrate dihydrate and Glycopyrronium salts were dissolved in Purified water. Hydroxypropyl methylcellulose, Sorbitol, Glycerol, Polysorbate, Polyoxyethylene Lauryl Ether and menthol were dissolved in water in a separate container under continuous stirring and mix for 10-15 min. The obtained solution was added to drug solution under stirring and mix for 10 min to obtain a clear, viscous dispersion. Aspartame was added to the mixture and mix for 5 min. Ethanol added to the mixture and mix for 5 min. Lemon mint flavour added to the dispersion and mix for 3 min. The resulting mixture was placed under vacuum to eliminate air bubbles. Poured the solution on Norfilm in required thickness by using Equipment: Mathis Lab coater (Model No: LTE-S, Mfr.: Werner Mathis AG, Switzerland). After uniform distribution of the dispersion, the film was dried at the temperature of 50-60° C. The film can be cut in required sizes/shapes.
The physical and mechanical properties of Glycopyrrolate oral films compositions of examples 1, 3 and 7 are evaluated which are given in the table below:

| | | Example 1 | Example 3 | Example 7 |
|---|---|---|---|---|
| S. | | Results | Results | Results |
| No. | Parameter | (Mean ± SD) | (Mean ± SD) | (Mean ± SD) |
| 1. | Thickness (μ) | 52 ± 3 | 68 ± 3 | 76 ± 3 |
| 2. | Disintegration (sec) | 24 ± 2 | 32 ± 2 | 35 ± 3 |
| 3. | Residual water content | 2.98 ± 0.32 | 3.7 ± 0.2 | 5.6 ± 0.4 |
| | (%) | | | |
| 4. | Young's Modulus (M | 61.25 ± 4.21 | 75.3 ± 3.2 | 72 ± 3 |
| | pa) | | | |
| 5. | Tensile strength (M pa) | 4.6 ± 0.4 | 6.2 ± 0.4 | 4.8 ± 0.2 |
| 6. | Tensile strain (%) | 3.8 ± 0.2 | 5.1 ± 0.3 | 5.2 ± 0.6 |
| 7. | Folding endurance | 44 ± 3 | 65 ± 4 | 36 ± 3 |
| 8. | Swelling index (%) | 38.6 ± 4.3 | 46.1 ± 3.3 | 57 ± 3 |
| 9. | Transparency (%) | 0.16 ± 0.03 | 0.23 ± 0.04 | 0.21 ± 0.03 |

SD—Standard deviation

1: A novel Glycopyrronium salts oral film composition comprising:
0.05% to 5% (w/w) of Glycopyrronium salts,
0.1% to 10% (w/w) of saliva stimulating agents,
0.5% to 60% (w/w) of film-forming agents,
0.05% to 10% (w/w) of buffering agents,
0.1% to 25% (w/w) of plasticizers, and
0.5% to 90% (w/w) of other pharmaceutically acceptable excipients.

2: The novel Glycopyrronium salts oral film composition as claimed in claim 1, wherein said Glycopyrronium salts are selected from bromide, acetate, benzoate, edisylate, oxalate, hydrogen sulfate and tosylate.

3: The novel Glycopyrronium salts oral film composition as claimed in claim 1, wherein said saliva stimulating agent is selected from citric acid anhydrous, malic acid, lactic acid, tartaric acid and ascorbic acid or mixtures thereof.

4: The novel Glycopyrronium salts oral film composition as claimed in claim 1, wherein said buffering agents are selected from citric acid, sodium carbonate, sodium citrate dihydrate, trisodium citrate dihydrate, ascorbic acid, acetic acid, tartaric acid, citric acid monohydrate, sodium bicarbonate, potassium carbonate, potassium bicarbonate, sodium phosphate dibasic, sodium phosphate tribasic, disodium phosphate dibasic, potassium citrate, potassium phosphate dibasic, potassium phosphate tribasic, tricalcium phosphate, calcium carbonate, calcium phosphate, carbonated calcium phosphate, magnesium carbonate, sodium hydroxide, magnesium hydroxide, potassium hydroxide, aluminium hydroxide or mixtures thereof.

5: The novel Glycopyrronium salts oral film composition as claimed in claim 1, wherein said film-forming agents are selected from hydroxypropyl methylcellulose grades, hydroxypropyl cellulose, Sodiumcarboxy methylcellulose, polyvinyl alcohol, polyvinyl acetate, hydroxyethyl cellulose, methylcellulose, ethylcellulose, polyvinyl pyrrolidone, polyethyleneoxide, carrageenan, gelatin, dextrin, polyethylene oxide, Starch, Pectin, Sodium alginate, guar gum, xanthan gum, carrageenan, glycerol monooleate, maltodextrin, tragacanth gum, sodium citrate dihydrate, polyvinyl alcohol-polyethylene glycol co-polymers, carboxylic acid containing polymers such as acrylic acid, methacrylic acid, esterified poly acrylic acid polymers, such as polyacrylic acid polymers lightly crosslinked with a polyalkenylpolyethers, methacrylate polymers, maleic acid copolymers, methacrylic acid-ethyl acrylate copolymers, methacrylic acid and methacrylate based polymers, and a methylmethacrylate copolymer or mixtures thereof.

6: The novel Glycopyrronium salts oral film composition as claimed in claim 1, wherein said plasticizers are selected from polyethylene glycols, propylene glycol, polypropylene glycols, polyethylene-propylene glycols, glycerol, glycerol monoacetate, diacetate or triacetate, triacetin, polysorbate, cetyl alcohol, sorbitol, sodium diethylsulfosuccinate, triethyl citrate, glycerine, tributyl citrate or mixtures thereof.

7: The novel Glycopyrronium salts oral film composition as claimed in claim 1, wherein other pharmaceutically acceptable excipients are selected from permeation enhancers, preservatives, humectants, antioxidants, superdisintegrants, taste modifying agents, colouring agents, stabiliser, thickening agents, emulsifiers, sweetening agents, flavouring agents.

8: The process for the preparation of Glycopyrronium salts oral film as claimed in claim 1, comprising the steps of:
(a) dissolving saliva stimulating agent, buffering agent and Glycopyrronium salts in purified water,
(b) adding additional film forming agents/polymers to the solution of step (b) under continuous stirring to form clear solution,
(c) adding plasticizers and other pharmaceutical acceptable excipients to the obtained solution of step (b) under continuous stirring to form clear solution/uniform dispersion,
(d) pouring the obtained solution of step (c) on inert support in required thickness, and
(e) drying the film at the temperature of 50-60° C. and cutting into required sizes/shapes.

9: The process for the preparation of Glycopyrronium salts oral film as claimed in claim 1, comprising the steps of:
(a) mixing stabilizer, plasticizer, film forming agent, taste masking agent in purified water under continuous stirring,
(b) adding solvent and additional taste masking agent to solution of step (a),
(c) dissolving saliva stimulating agent and buffering agent in purified water separately,
(d) adding Glycopyrronium salts to buffer solution of step (c) under continuous stirring,
(e) adding Glycopyrronium salts solution to the solution of step (b), and
(f) placing obtained mixture under vacuum, casting as a uniform layer onto an inert support, drying in an oven and cutting into required sizes/shapes.

10: The process for the preparation of Glycopyrronium salts oral film as claimed in claim 1, comprising the steps of:
(a) adding thickening agent in purified water under continuous stirring to form clear liquid,
(b) adding buffering agent, antioxidant, plasticizer, film forming agent under continuous stirring,
(c) dissolving saliva stimulating agent and additional buffering agent in purified water separately,
(d) adding Glycopyrronium salts to buffer and dissolving it under continuous stirring,
(e) adding Glycopyrronium salts solution to the solution of step (b),
(f) adding solvent to the solution of step (e) under continuous stirring to form uniform mixture, and
(g) placing obtained mixture under vacuum, casting as a uniform layer onto an inert support, drying in an oven and cutting into required sizes/shapes.
